(19) United States
(12) Patent Application Publication
(10) Pub. No.: US 2006/0014301 A1
(43) Pub. Date: Jan. 19, 2006

(54) Antibody-based system for detection of differential protein expression patterns

(75) Inventor: Ira Leonard Goldknopf, The Woodlands, TX (US)

Correspondence Address: ELIZABETH R. HALL, 1722 MARYLAND STREET, HOUSTON, TX, 77006, US

(73) Assignee: Power3 Medical Products, Inc., The Woodlands, TX (US)

(21) Appl. No.: 11/179,740

(22) Filed: Jul. 12, 2005

Related U.S. Application Data
(60) Provisional application No. 60/587,446, filed on Jul. 13, 2004.

(51) Int. Cl. G01N 33/543; G06K 9/00

(52) U.S. Cl. 436/518; 382/128

(57) ABSTRACT

The present invention is a kit and method for identifying the presence or absence of a protein expression profile that is known to be associated with a particular disease or an altered biological state. The method is based on the combination of a known protein expression pattern biomarker with the use of an antibody-based detection system. The images of two antibody-based detection systems are compared by an overlay procedure to determine protein expression patterns in biological samples.

### CROSS REFERENCE TO RELATED APPLICATIONS

This application claims priority to U.S. Provisional Patent Application Ser. No. 60/587,446 filed Jul. 13, 2004 and entitled “Antibody-based System for Detection of Differential Protein Expression Patterns” by inventors Ira L. Goldknopf, et al.

### BACKGROUND OF THE INVENTION

1. Field of the Invention

The invention relates to a multiple antibody-based assay system for identifying the existence of protein expression patterns in biological samples, wherein the presence of the protein expression pattern can be indicative of an alteration in some biological process in an organism, including disease in a human.

2. Description of the Related Art

A biomarker is commonly defined as a substance present in a biological sample that is characteristic of the presence of an identifiable condition or biological state. As applied to human disease, a biomarker is a substance that can be detected in either body fluids or cells and tissues that is predictive of either the presence or absence of disease or an alteration in normal physiology. Detection of disease biomarkers has been an active area of research in the last decade with many different individual biomarkers having been identified. In addition to detection of disease, biomarkers are used in other areas of biology. For example, drug use has been detected by the detection of drug metabolites and environmental insults can be identified by the stimulation or inhibition of certain enzymes in organisms or in environmental media. In that case, the biomarker is used as an indicator of an alteration in a biological process or condition.

Biomarkers of disease have included the well established identification of estrogen and progesterone receptors with progression of breast cancer, insulin levels as a biomarker of diabetes, and serum liver enzyme levels as biomarkers of liver damage, but these are only a few of the hundreds of different biomarkers that have been linked to some type of human disease. The focus of biomarker research, however, has been on identification of single biomarkers of a particular disease or altered biological state.

Protein levels can be detected analytically through a variety of methods. One of the popular methods employs use of antibodies specific to a given proteinaceous antigen. Antibody-based analytical methods have been widely used in medicine for decades and have permitted both qualitative and quantitative detection of the presence of a protein in body fluids. Protein detection through use of antibodies in biological samples is well known in the art and includes application of methods such as radioimunoassay (RIA), stains, and enzyme-linked immunosorbant assay (ELISA).

Radioimunoassay is based on the principle of competitive inhibition of the binding of a radio labeled antibody with an unlabeled antigen. Radio labeled antibody is bound to a surface and the binding is displaced through contact of the antibody with unlabeled antigen (protein). Antigen-antibody complexes are separated from unbound antigen and the amount of radioactivity of a sample is measured as a way to determine the presence or absence of unlabeled antigen (protein). Any method can be used to separate antigen-antibody complexes present in a sample. Common methods include a double antibody technique wherein antigen-antibody complexes are precipitated out of solution using a second antibody that binds to the first antibody. Another method that can be used is a dextran activated charcoal technique where the addition of charcoal and immediate centrifugation results in separation of unbound antigen. Such radioimunoassay methods have been described in numerous patents as methods to identify proteins in samples (see for example U.S. Pat. Nos. 5,366,859; 4,594,319; 4,591,573; 4,543,340; 4,489,166; 4,438,209; 4,438,207).

Another commonly employed antibody-based detection method contemplated by the instant invention is use of ELISA. In this method, an enzyme tag is attached to an antibody instead of a radioactive label. In this method, enzyme-linked antibodies that are specific for the proteins to be detected would be used. After recognition/contact of the antibodies with the proteins to be detected, excess antibody is removed from the sample. The ELISA method can also involve use of a second antibody that is linked to the enzyme. The detection of a protein is indicated by the presence or absence of enzymatic activity in the sample. Such ELISA methods have been described in numerous patents as methods to identify proteins in samples (see for example U.S. Pat. Nos. 6,350,584; 6,270,985; 6,258,549; 6,204,367; 5,985,545; 5,776,671; 5,712,104; 5,202,264; 4,764,459; 4,661,445).

In most cases, such antibody methods are directed towards identification of single proteins in samples. Even the methods designed to detect more than one protein in a sample do not allow one to measure whether the expression of the detected proteins have been up-regulated or down-regulated.

There is a need for antibody-based assay systems that can compare the expression of multiple biomarkers in standard solutions, control samples and patient samples and through such comparison detect patterns of biomarkers that are diagnostic of disease.

There is also a need for a multiple antibody-based assay system that does not require sophisticated equipment and laboratory facilities.

### SUMMARY OF THE INVENTION

The present invention is a method for identifying the presence of a protein expression pattern that is characteristic of a biological sample from an organism expressing an altered biological state which comprises: a) coating a first and a second solid surface with a plurality of antibodies, wherein said antibodies are antibodies that are reactive to a set of proteins characteristic of a protein expression pattern found in a biological sample taken from an organism having an altered biological state; b) contacting the first solid surface with a standard sample; c) washing the standard-contacted first solid surface to remove all protein that is unreacted with the coated antibodies; d) contacting the second solid surface with a biological sample; e) washing the sample-contacted second solid surface to remove all protein that is unreacted with the coated antibodies; f) creating a first digital image of the washed first solid surface and a second digital image of the washed second solid surface; g) assigning a first color to the first digital image and a second color to the second digital image, wherein an intensity of the first and second colors are proportional to a concentration protein bound to the antibodies; h) overlaying the first and second digital images; and i) analyzing the overlaid images to determine if the biological sample was from an organism having the altered biological state.

Another object of the present invention is an assay method for an altered biological state comprising: a) coating a first and a second solid surface with a plurality of antibodies, wherein each antibody reacts with an antigenic determinant in a protein associated with an altered biological state; b) contacting the first solid surface with a standard sample; c) contacting the second solid surface with a biological sample; d) washing unreacted protein from the standard-contacted first solid surface and the sample-contacted second solid surface; e) staining the washed first solid surface with a first reporter molecule and the washed second solid surface with a second reporter molecule, wherein the first reporter molecule and the second reporter molecule are visually distinguishable from each other; f) overlaying the first and second stained solid surfaces; and g) analyzing the overlaid stained surfaces to determine if the biological sample was from an organism having the altered biological state.

Yet another object of the present invention is a kit for screening biological samples to determine the biological samples relationship to an altered biological state comprising: a) a first solid surface coated with a plurality of individual antibody spots, wherein each antibody reacts with an antigenic determinant of a protein associated with an altered biological state; b) a second solid surface coated with a plurality of individual antibody spots, wherein a number of the antibody spots on the second solid surface are substantially similar to the antibody spots on the first solid surface; c) a standard sample; e) a first reporter molecule; f) a second reporter molecule; and g) means for analyzing an overlay of the first coated solid surface reacted with the standard sample and the first reporter molecule with the second coated solid surface reacted with a biological sample and the second reporter molecule.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 illustrates a test of sample staining and image analysis according to the present invention;

FIG. 2 illustrates one embodiment of a linear array of an antibody-based system for detection of differential protein expression patterns;

FIG. 3 illustrates a hypothetical assay system pursuant to the present invention for assessing the risk of a heart attack;

FIG. 4 illustrates an embodiment of a star-shaped array of an antibody-based system for detection of differential protein expression patterns;

FIG. 5 illustrates an embodiment of the antibody-based assay system shown in FIG. 3 for determining the risk of breast cancer;

FIG. 6 illustrates an embodiment of the array system shown in FIG. 3 for comparing differential protein expression patterns for a right and a left breast of an individual; and

FIG. 7 illustrates an embodiment of the antibody-based system of the present invention designed to determine the severity or staging of a disease.

### DESCRIPTION OF THE PREFERRED EMBODIMENTS

The present invention is a method and a kit for the detection and/or quantification of one or more biomarkers using an antibody-based system. The method and kit of the present invention can be used to detect differential quantities of any antigenic material, although the most common antigens detected will be proteinaceous material. Proteinaceous material as defined herein includes both proteins and peptides. Throughout the description of the invention below the terms “protein,” “peptide” and “proteinaceous material” will be used interchangeably and are meant to include proteins and peptides.

Typically, the present invention is a determination of protein expression profile differences among biological samples taken from patients with and without disease or altered biological states. In the context of the present invention a “disease” or “disease state” is a condition wherein an individual or patient exhibits a known set of symptoms or biological changes and would include, but not be limited to, cancer (e.g., breast cancer, prostate cancer, brain cancer, uterine cancer, and ovarian cancer), neurodegenerative disease (e.g., Alzheimer's disease, ALS, and Parkinson's disease), and autoimmune disease (e.g., rheumatoid arthritis, systemic lupus erythematosus, and multiple sclerosis). In the context of the present invention, an “altered biological state” is any situation where the individual's or patient's normal biological function has been shown to be different as compared to the function that individual had known previously, or which have been identified as normal in a population of individuals.

The method is based first on the identification of patterns of protein expression that are characteristic of a disease state. The identification of these disease-specific protein expression profiles are then used as the basis for construction of a disease-specific antibody-based kit for detection of the disease-specific protein expression profiles in patient samples.

Antibody methods are well known in the art as methods for detecting protein and peptide expression in biological samples. Any method that allows for detection of an antigen (e.g., a peptide or protein) with an antibody is contemplated by the present invention including radioimunoassay (RIA), enzyme-linked immunosorbant assay (ELISA), Western blotting, and immunofluorescence. These methods have been described in standard texts of immunological techniques (see for example, Immunology, J. Kuby (ed.), 1991. W.H. Freeman and Co.: New York, pp. 135-156).

Typically, the antibody-based assays described have been used to detect single biomarkers of disease, not a plurality of biomarkers in the same sample, wherein the plurality of biomarkers is shown to be indicative of the presence or absence of disease or an altered biological state. Previous research has demonstrated the validity of identifying protein expression patterns that are characteristic of disease states in tissues from patients, as for example U.S. Pat. No. 6,855,554. With the disease-specific protein expression pattern identified, the disease biomarkers will then be applied to construction of a kit for detection and diagnosis of disease using antibody-based methods.

In the context of the present invention, the term “antibody” refers to any antibody-like molecule that has an antigen binding region and would include antibody fragments such as Fab′, Fab, F(ab′)2, single domain antibodies, Fv, scFv, and the like. Techniques for preparing and using various antibody-based constructs are well known in the art, as are means for preparing and characterizing such antibodies (see for example Antibodies: A Laboratory Manual, Cold Spring Harbor, 1988).

In the present invention, a kit is prepared by first raising antibodies to the proteins identified as being biomarkers of the disease. In some cases the proteins may have been shown to be up-regulated and differentially expressed while others may have been shown to be down-regulated and differentially expressed. This differential pattern of protein expression will be used as a basis for development of the kit of the instant invention, wherein each disease will be known to have its own unique disease-specific differential protein expression pattern.

In one arrangement of the kit of the present invention, one or more antibodies targeted to antigenic determinants of proteinaceous material will be spotted onto the surface of a solid support. The solid support would include, but not be limited to, any plastic surface that is amenable to antibody binding, such as the wells of standard 96 well plates. Such methods may also include micro arrays, or antibodies or proteins positioned on a glass slide or silicon chip. It is contemplated that the antibodies will be prepared and presented on a solid surface in a specific array or pattern. These arrays or can be in any pattern including without limitation a linear, circular, triangular, pyramidal, arrow, or star-like array.

As the antibodies used will each recognize their specific antigen, incubation of the spot patterns with the samples will permit attachment of the specific proteinaceous material containing the antigenic determinant to the antibody. Bound antigen can be reported by any of the known reporter techniques including sandwich ELISA with an HRP-conjugated second antibody also recognizing the specific protein, pre-conjugating fluorescent dyes such as the cyanine dyes Cy3 or Cy5 to the proteins in the samples to provide a photostable fluorescence to the bound protein, or biotinylating the proteins in the samples and using an HRP-bound streptavidin reporter. The HRP can be developed using a chemiluminescent, fluorescent, or colorimetric reporter. Other enzymes such as luciferase or glucose oxidase, or any enzyme that can be used to develop light or color can be utilized at this step. The patterns seen in intensity of color, light emission, or radioactivity levels will be used to qualitatively and quantitatively identify the patterns of protein expression in biological samples.

Alternatively, the sample proteins may be spotted on a solid matrix and then reacted with an antibody directed to a biomarker antigen. The binding of the antibody may be detected by using biotinylated or fluorophore-conjugated antibodies, or by using a reporter-labeled antibody as a second antibody. A wide variety of reactions and reporter systems have been described in the literature and any one of these systems are applicable to the method and kit of the present invention.

The method can comprise many different specific steps using many different antibody-based tools known to those skilled in the art. In general, the method comprises: a) coating a first solid surface with a plurality of antibodies, wherein said antibodies are antibodies that have been targeted to proteins that comprise a protein expression pattern indicative of an altered biological state; b) coating a second solid surface with the same plurality of antibodies present on the first solid surface; c) contacting the first solid surface with a standard sample, wherein the standard sample contains known levels of the proteins of the protein expression pattern or levels of the proteins of the protein expression pattern that are not associated with the altered biological state; d) contacting the second solid surface with a biological sample; e) contacting the first solid surface with a reporter molecule, wherein the binding of the reporter molecule is proportional to the quantity of the protein bound to each antibody; f) contacting the second solid surface with the reporter molecule; g) creating digital images of the first and second solid surfaces; h) assigning the images of the first solid surface a first color and the images of the second solid surface a second color, wherein the two colors form an additive color when the two colors are combined; i) overlaying the images of the first and second solid surfaces; and j) analyzing the overlaid images to assess the likelihood that the biological sample was taken from an organism having the altered biological state.

The present invention is also a kit that can be used to identify if a biological sample is taken from a patient having an altered biological state. In one embodiment the kit comprises: a) a first solid surface wherein the surface is coated with a plurality of antibodies, wherein the antibodies are antibodies that have been targeted to proteins that comprise a protein expression pattern indicative of an altered biological state; b) a second solid surface wherein the surface is coated with the same plurality of antibodies; c) a standard sample, wherein said standard sample is a sample that contains known levels of the proteins of the protein expression pattern or levels of the proteins of the protein expression pattern that are not associated with an altered biological state; d) an optional test sample from an altered biological state, wherein the test sample contains a protein expression pattern indicative of the altered biological state being tested for with the kit; and e) a reporter molecule. Alternatively the method and the kit may use stained reporter molecules. In that case, a first stain will be used for one reacted array or the first solid surface and a second stain will be used for the second reacted array or the second solid surface that is to be compared with the first reacted array, the first and the second colors being additive to create a third color that is visually distinguishable from the first and second color.

In addition to use for detecting patterns of disease biomarkers in biological samples, the kit of the instant invention can be used to detect the presence of many different types of compounds in biological samples, whenever a pattern of protein expression has been identified as characteristic of exposure to a particular compound. Therefore, applications of the kit and the method of the present invention would also include but not be limited to detection of environmental stimulants and drug metabolites.

The following non-limiting examples are provided in order to better illustrate the present invention. Although the examples given utilize specific antibodies spotted on a solid matrix, one skilled in the art would know how to construct the antibody-based system by spotting the samples and detecting the proteins in those samples using antibodies targeted to any specific proteins of interest.

### EXAMPLE 1

### Image Analysis

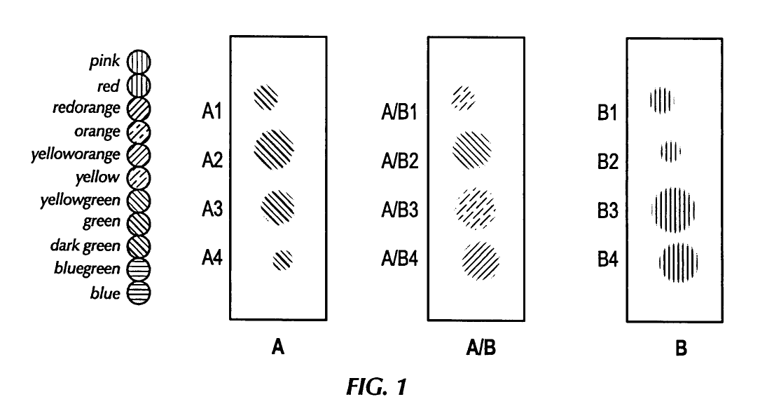

FIG. 1 represents a feasibility test of sample staining and image analysis. The spots A1-A4 represent spots of rabbit serum, where spots A2 and A3 have an equal amount of rabbit serum that is a significantly greater concentration of rabbit serum than is in spots A1 and A4. Similarly, the spots B1-B4 also represent spots of rabbit serum. The concentration of rabbit serum in spots B1 and B2 are equal to each other and to the concentration of rabbit serum in A1 and A4, while the concentration of rabbit serum in spots B3 and B4 are equal to each other and to the concentration of rabbit serum in A2 and A3.

Each of the serum spots (i.e., A1-A4 and B1-B4) was stained with a FITC-conjugated goat anti-rabbit serum. Spots A/B1-A/B4 show the results of overlaying a digital image of spots B1-B4 with a digital image of spots A1-A4.

Placing the slide having the stained serum spots on the imaging platform of a FX-PRO Laser Scanner and scanning an image of the stained spots into the PDQUEST software program initiates one embodiment of the image analysis procedure of the present invention.

The process of image analysis for the stained protein spots begins by cropping the images to be analyzed and filtering them to eliminate the stain precipitate. The cropping must be done such that the spots can be compared using the Multichannel viewer option in PDQUEST. This is generally accomplished by rotating the image and/or adjusting the cropped image horizontally or vertically. The images to be compared must be the same size as measured in pixels. The PDQUEST software has an image option that allows the user to reduce or expand the file size without distorting the image.

Two stained protein spots are selected for comparison of their protein concentration and each image is assigned a different color. The Multichannel viewer produces images with black backgrounds and colored protein patterns. The colors assigned to the first stained protein spot (i.e., the first color) and the second stained protein spot (i.e., the second color) are typically at different ends of the color spectrum so that if an equal intensity of the colors are added together one would get a third color (an additive color).

The two colored protein spot images are overlaid, either physically or electronically. Since overlaying two distinctly different colored stained protein spots result in visually apparent color variations in the overlaid images, slight corrections in alignment patterns are readily made. In fact, the manual alignment of the two protein spots to maximize the amount of the additive color seen in the overlaid spot images is very effective. Alternatively, one can select to have the digital images electronically aligned to optimize the additive color.

The resulting color of each of the overlaid protein spots is quite informative. If a stained protein spot in one gel is overlaid with another stained protein spot to give the additive color, then the protein concentration in the two stained protein spots is similar. On the other hand, whenever one stained protein spot is overlaid with another stained protein spot to yield a non-additive color closer to the spectra of the first or second color, and then the protein concentration in the two stained protein spots is different. If the resulting color of the overlaid spots is closer to the wavelength of the color assigned to the first stained spot, the concentration of the stained protein in the second spot is lower than in the first spot. Whereas, if the resulting color of the overlaid spots is closer to the wavelength of the color assigned to the second spot, the concentration of the stained protein in the second spot is greater than in the first spot.

Alternatively, the color of the overlaid spots may be measured at three wavelengths (i.e., the wavelengths of the first color, the second color, and the additive color). By comparing the three-wavelength measurements a quantitative comparison of the stained protein in each of the overlaid spots can be determined.

In FIG. 1 where spots A1-A4 are green and spots B1-B4 are red the color of the overlaid spots indicate the comparative protein concentrations in the two overlaid spots. For example, A/B1 and A/B3 are yellow illustrating the overlay of two spots of an equal concentration of rabbit serum stained with FITC-labeled goat anti-rabbit serum. In contrast, A/B2 is a yellowish green indicating that spot A2 had more rabbit serum than spot B2 and A/B4 is a reddish orange indicating that spot A4 had less rabbit serum than spot B4.

### EXAMPLE 2

### Array for the Detection of the Presence or Absence of a Disease State

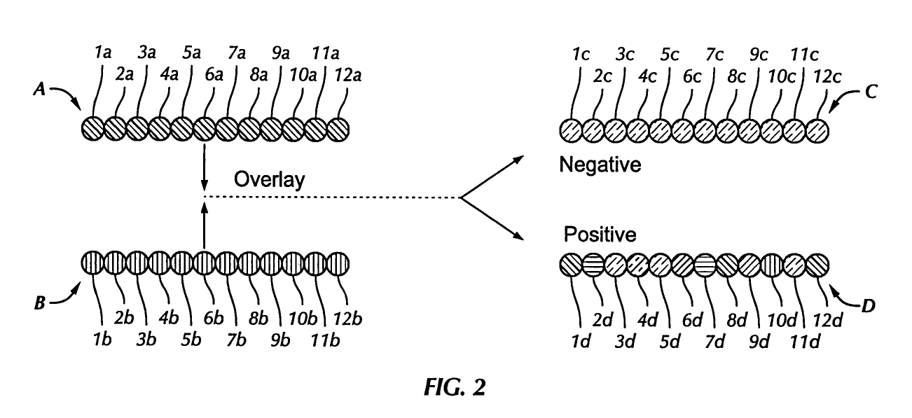

One embodiment of the antibody-based system utilizes a linear array of antibodies. Thus, antibodies to the differentially expressed proteins are arranged in a single line of spots as shown in FIG. 2.

One linear array of spots (array A, spots 1a-12a, seen in FIG. 2) is exposed to an unknown sample from an individual that may or may not have a disease or altered biological state; whereas an identical pattern of spots (array B, spots 1b-12b, seen in FIG. 2) is exposed to a sample indicative of a biological state that has not been altered. Such a sample may be a mixture of “normal” concentrations of the proteins included in the protein expression pattern, or it may be a control sample (a sample of a disease-free individual or one indicative of the biological state that has not been altered). Optionally, one or more additional arrays (not shown) are exposed to various concentrations of the target protein standards, or normal or control biological samples, or biological samples from organisms known to have the disease or altered biological state of interest.

The presence or absence of specific protein binding is determined in each of the linear arrays. The binding of a specific protein can be determined in a number of different ways. For example, a second antibody, with a coupled reporter molecule, may be used to bind to another antigenic determinant within the protein. Alternatively, various concentrations of the target protein standard labeled with a reporter molecule may be added to the reaction of the sample and the specific antibody. The labeled standard protein would compete with the protein in the sample for binding with the antibody, leading to a decrease in standard binding when high concentrations of the protein are in the sample in a mechanism similar to that employed in radioimunoassays (RIAs).

In order to detect a pattern of binding, the assay will incorporate reactions that are based on colors/stains that can be distinguished from each other visually, or reactions that provide varying intensities of product where the intensity of the reaction product can be digitized and distinguished as variations in color or in shades of gray. Each reacted linear array is assigned a different color, the intensity of the color being based on the degree of antigen binding. The colors assigned to the control array (first color) and the test array (second color) will be at different ends of the color spectrum so that if an equal intensity of the colors are added together one would get a third color (the additive color).

For example, if the control array is reacted with standards and the test array is reacted with the same standards, the two reacted arrays would have substantially identical protein bound to the arrays. If the product intensity of the reacted control array is assigned red and the product intensity of the sample array is assigned green, the overlay of the red over the green would yield the additive color yellow. However, if the control array is reacted with standards and the test array is reacted with a patient sample having different quantities of the antigenic proteins than the standards, then the overlay of the reacted control array and the test array would produce variations in color.

Since the selected proteins of interest are not differentially expressed in fully normal individuals or in a sample that is known to be unaffected by disease or is unaltered, the overlay of the reacted control and normal sample arrays would generally yield a uniform additive color (as seen in spots 1c-12c in FIG. 2). In the case of individuals with disease, however, the pattern will be multi-colored (as seen in spots 1d-12d seen in FIG. 2) as one or more of the biomarker proteins would be absent or present in a different quantity.

The biological samples employed with the present invention can be samples from individuals suspected of having an altered biological state or disease (unknown samples), control samples, or standard solutions with known proteins contained therein. A “control” sample can be a sample from an individual known not to have the altered biological state or to be disease-free, or the “control” sample can be one from the same individual but representative of cells or tissues not affected by the altered biological state or disease. An example of such a control sample would be the nipple aspirate fluid sample from a breast that is known to be non-cancerous and comparing it with the unknown sample of the nipple aspirate fluid from a breast suspected of being cancerous.

Standard solutions may be used in situations where a quantitative level of protein expression has been determined and a solution containing the “normal” or “standard” levels of each protein can be used. Standard solutions are particularly useful where biomarkers are only present when the altered biological condition is present, or alternatively are only absent when the altered biological condition is present.

Whenever the quantitative levels of expression of a protein are important, one may develop a standard curve by reacting different known quantities of the standard with the antibody spot to determine the resulting color when the selected protein is present in a greater or lesser quantity than in the control sample. Therefore, the present invention is a method for identifying the presence of a protein expression pattern that is characteristic of a biological sample of an altered biological state.

Detecting Myeloblastic Leukemia

Variations in the mRNA levels for different interferons (IFNs) has been observed in normal versus leukemic human blood leucocytes (Hiscott, J. et al. 1984. Philos. Trans. R. Soc. Lond. B. Biol. Sci. 307(1132):217.) For example, all cases of myeloblastic leukemia examined showed a high expression of INF-alpha 14. Thus, an antibody-based assay system would be developed wherein antibodies to various alpha-, beta- and gamma-interferons would be arranged on a solid matrix and samples and standards reacted with these antibodies to detect the unique patterns of expression in myeloblastic leukemia.

Detecting the Risk of Heart Attack

Recent research has identified a pattern of cardiac biomarkers that are indicative of the risk of heart attack (Wiviott, S. D. et al. 2004. Circulation 109:565-567). Using the identity of these known biomarkers for risk of heart attack, a kit can be constructed that is used to detect the unique pattern of these enzymes that is characteristic of a high risk for heart attack. The increased expression of C-reactive protein and brain natriuretic peptide and the decreased expression of creatinine kinase MB and troponins has been shown to be indicative of a high risk of heart attack.

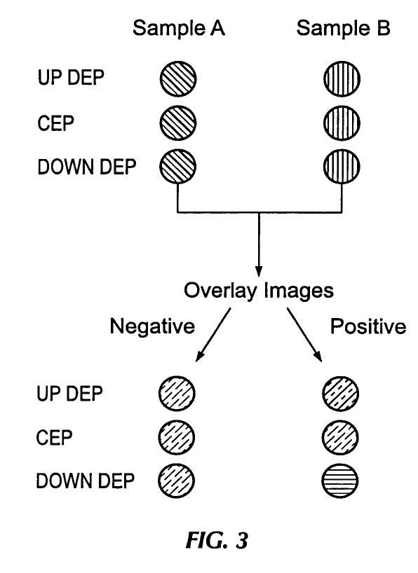

Therefore, an antibody-based assay system such as shown in FIG. 3 could be used to indicate a high risk of heart attack in particular patients. One embodiment of a kit developed pursuant to the present invention would have linear arrays of antibodies directed to a known up-regulated differentially expressed protein (UP-DEP, e.g., C-reactive kinase), a constitutively expressed protein (CEP), and a down-regulated differentially expressed protein (DOWN DEP, e.g., creatinine kinase MB).

The prepared linear arrays would then be reacted with samples. In FIG. 3, Sample A is a “normal” individual without an increased risk of heart attack and Sample B is a patient that is negative for an increased risk of heart attack (see the overlay result labeled Negative) or a patient with an increased risk of heart attack (see the overlay results labeled Positive).

### EXAMPLE 3

### Expression of Protein Array with Up-Regulated and Down-Regulated Proteins

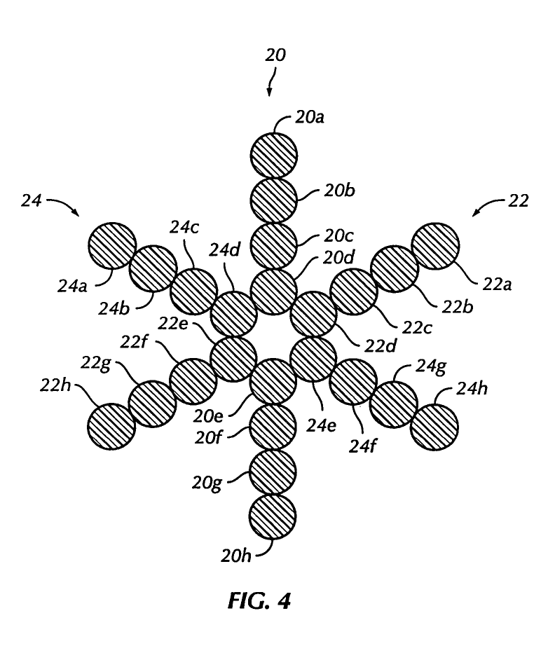

Another embodiment of the present invention is shown in FIG. 4. This embodiment takes advantage of known up-regulated biomarkers and down-regulated biomarkers. For example, this embodiment may be used for the detection of breast cancer using a set of proteins that are known to be constitutively expressed, proteins known to be consistently up-regulated and proteins known to be consistently down-regulated.

As described above, antibodies targeted to specific proteins are spotted on a solid matrix in an array. One such array is the star-like pattern shown in FIG. 4, where known constitutively expressed proteins are spotted in the vertical line 20 (spots 20a-20h), consistently up-regulated proteins are spotted in line 22 (spots 22a-22h), and consistently down-regulated proteins are spotted in line 24 (spots 24a-24h).

As previously described identical star-like arrays are reacted with a variety of samples, including without limitation a control sample, a series of standards, a sample taken from a patient or organism having a known disease or altered state, and an unknown sample. The reacted arrays are then stained with a reporter molecule that provides a colored reaction product or a reaction product providing a quantitative intensity that can be digitized and assigned a color.

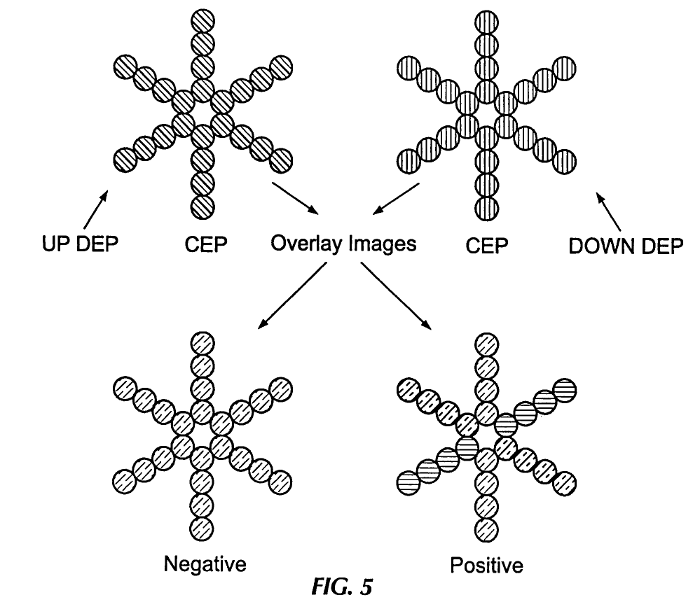

When the reacted colored arrays of two samples are overlaid, the resulting colors of the spots will provide information as to the nature of an unknown sample. For example, a single additive color (e.g., Negative in FIG. 5) will result from overlaying arrays reacted with samples that are non-disease or non-altered, such as two control samples or a normal sample and a control sample. However, overlays of control samples and samples having different quantities of the protein biomarkers will exhibit a variety of colors (e.g., Positive in FIG. 5).

Unknown samples containing differentially expressed up-regulated proteins will give varying colors when overlaid over a control sample pursuant to the present invention. The resulting color of the overlaid spots for the UP DEPs, where the unknown sample is stained a first color (e.g., red) and the control sample is stained a second color (e.g., green), will vary along a spectrum proceeding from yellow to red. When the red-stained unknown sample is overlaid with the green-stained control sample the resulting color will not be yellow (i.e., the additive color of red and green) but will be a color having a wavelength closer to the color assigned to the unknown sample (e.g., orange). In fact, the greater the quantity of the up-regulated protein in the unknown sample the further the resulting color will be shifted towards the wavelength of the first color (i.e., red).

Similarly, unknown samples containing differentially expressed down-regulated proteins will give varying colors when overlaid over a control sample pursuant to the present invention. The resulting color of the overlaid spot, where the unknown sample is stained a first color (e.g., red) and the control sample is stained a second color (e.g., green), will vary along a spectrum proceeding from green to yellow. When the red-stained unknown sample is overlaid with the green-stained control sample the resulting color will not be yellow (i.e., the additive color of red and green) but will be a color having a wavelength closer to the color assigned to the control sample (e.g., light green). In fact, the less the quantity of the down-regulated protein in the unknown sample the further the resulting color will be shifted towards the wavelength of the second color (i.e., green).

Different patterns of coloration are indicative of a particular altered state or disease, or even the severity or stage of a particular disease. Different patterns may also indicate different syndromes having different treatment regimes so that physicians can utilize the test results to select treatment protocols.

Detection of Breast Cancer

Using proteomic analysis methods, 8 proteins have been identified as consistently up-regulated (UP DEP) in breast cancer and 8 proteins have been identified as consistently down-regulated (DOWN DEP) in cancerous breast tissue. In order to test for the presence of these biomarkers and their pattern of expression in patients suspected of having disease, breast ductal fluid samples were collected by nipple aspiration.

Each nipple aspirate fluid (NAF) sample was diluted with the addition of cold RPMI buffer, but Tris-buffered isotonic saline, or any other appropriate buffer solution may be used. The diluted NAF was aliquoted into 1.5 ml microfuge tubes in 100 microliter portions and frozen in liquid nitrogen before analysis.

The table below (Table 1) lists the panel of differentially expressed protein biomarkers (UP DEP and DOWN DEP) that have been identified in nipple aspirate fluid of breast cancer patients.

Using the proteins listed in Table 1, antibodies will be raised using standard methods, or purchased from commercial vendors. Antibody-based methods will then be used to determine whether breast cancer is detected in the nipple aspirate fluid samples collected.

TABLE 1

| Protein Spot Number | Identity of Protein |
|---|---|
| | Up-regulated in Cancerous Breast |
| 1 | RAB 3D |
| 2 | Synaptosomal associated protein 23 |
| 3 | Neuregulin |
| 4 | Cytokeratin 19 |
| 5 | Sorting Nexin |
| 6 | Fibulin |
| 7 | Follistatin |
| 8 | Alpha Actinin |
| | Down-regulated in Cancerous Breast |
| 1 | GST-mu³ |
| 2 | Visinin |
| 3 | PP2A |
| 4 | Calnexin |
| 5 | Retinol Binding Protein |
| 6 | Apolipoprotein A-IV |
| 7 | HLA-A |
| 8 | MUC 4 |

As discussed above, the pattern of protein expression detected will be visualized by methods such as color differentiation. When two different colors are used to distinguish normal samples from those samples with disease, the patterns detected will result in identification of the disease state of the sample. For example, FIG. 5 shows how the two color image overlay procedure can result in detection of a breast cancer protein expression pattern.

In FIG. 5, two samples, A and B, are shown, where sample A is a NAF sample of an unknown disease state and sample B is a NAF sample from a breast known to be cancer-free. When the images are overlaid on each other after antibody-based detection and application of a two coloration method, sample A would be identified as disease-free if the resulting color of the proteins expressed is shown to be of a uniform color (e.g., as labeled Negative in FIG. 4). If, however, sample A is identified as being likely to be associated with breast cancer, the pattern would be like the pattern labeled Positive in FIG. 5, where there are a mixture of colors or shades of color in the protein spots analyzed.

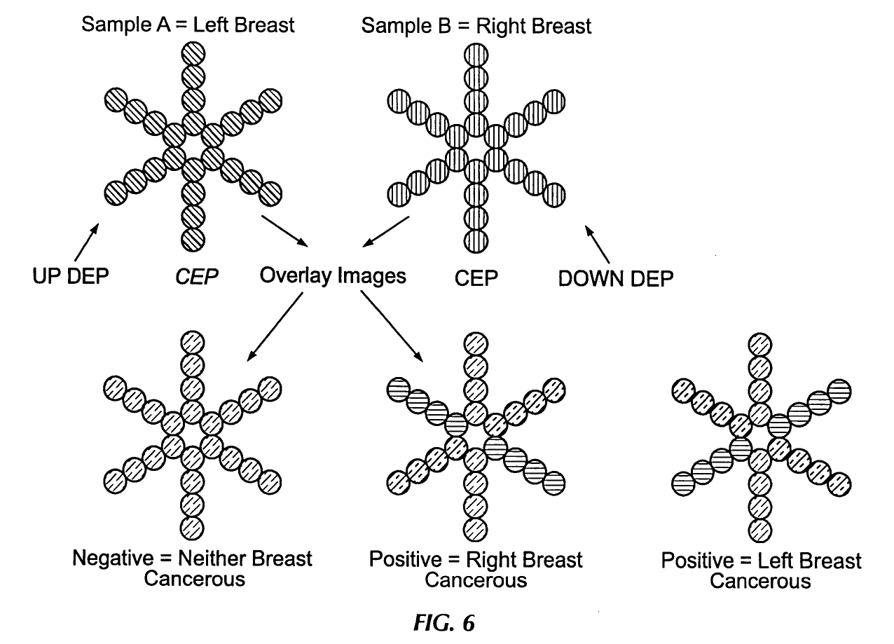

This process may also be used to assess the risk of breast cancer in a patient by comparing the protein expression pattern of the NAF of the right breast with the protein expression pattern of the NAF of the left breast, as shown in FIG. 6. In an individual that does not have breast cancer or an increased risk of breast cancer the overlay of the NAF samples of the right and left breast will give a uniform additive color.

However, where one of the breast either has breast cancer or is at an increased risk of breast cancer, the color pattern exhibited by the overlay of arrays reacted with the right breast NAF and the left breast NAF will vary in coloration. If the right breast is cancerous or at risk for breast cancer the pattern labeled Right Breast Cancerous in FIG. 6 will be exhibited. On the other hand, if the left breast is cancerous or at risk for breast cancer the pattern labeled Left Breast Cancerous in FIG. 6 will be exhibited. This assay allows an individual to serve as his/her own control sample and negates hormonal variations in the protein expression patterns of the samples.

### EXAMPLE 4

### Array to Detect the Stage or Severity of a Disease

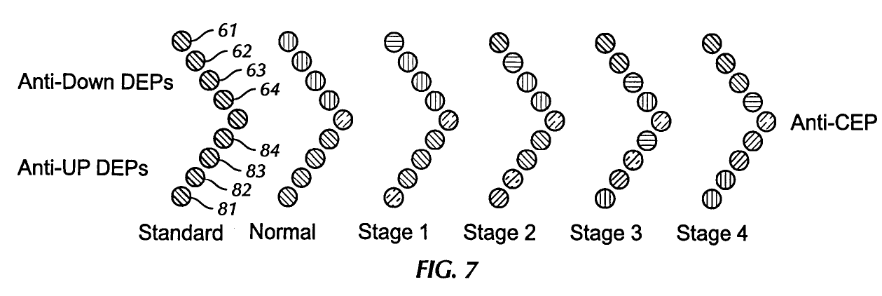

The embodiment of the present invention illustrated in FIG. 7 is used to indicate the severity of a disease or the phase or stage of a particular disease.

FIG. 7 illustrates one embodiment of a kit having antibodies directed to down-regulated differentially expressed proteins (Down DEPs) positioned in a descending right diagonal of four spots (spots 61-64 in FIG. 7). Antibodies to biomarkers associated with increasingly progressed disease are position in descending order (i.e., spot 61 is an antibody to a biomarker that disappears early on in the onset of the disease while spot 63 is an antibody to a biomarker associated with a more advanced stage of the disease).

Similarly, antibodies to up-regulated differentially expressed proteins (UP DEPs) are placed in an ascending right diagonal (spots 81-84 in FIG. 7). Antibodies to markers for increasingly progressed disease are place in ascending order (i.e., spot 81 is an antibody to a biomarker that appears early on in the onset of the disease while spot 83 is an antibody to a biomarker associated with a more advanced stage of the disease). Where the diagonal of the antibodies to the DOWN DEPs meet with the diagonal of the antibodies to the UP DEPs, an additional spot of an antibody to a constitutively expressed protein (CEP) is place for matching resultant product intensities to the standard pattern.

The standard sample comprises a known quantity of a CEP corresponding to a statistically validated mean quantity for control samples, an amount of the UP DEPs corresponding to a statistically validated quantity of the UP DEP proteins seen at the 95% upper confidence limit for control samples, and a quantity of the DOWN DEP proteins corresponding to a statistically validated quantity of the DOWN DEPs seen at the 95% lower confidence limit for control samples.

A first array is reacted with the standard sample and is color coded as a first color (see FIG. 7). A second array is reacted with the test sample and color coded as a second color (not shown). The intensity of the reaction product for the CEP second color spot of the test sample is set to match the intensity of the corresponding CEP first color spot of the standard sample.

When the test sample is normal, overlaying the color product of the reacted array would provide an array such as that labeled Normal in FIG. 7. For a normal test sample, the Down DEPs of the test sample would all be somewhat higher in intensity than the standard and be shifted towards the second color and the UP DEPs would be somewhat lower in intensity than the standard and be shifted towards the first color. The CEP would be equal in intensity with the standard and be an intermediate color between the first and second colors.

As the test sample is associated with increasingly severe disease, the color patterns of the overlays would shift to the reverse of that exhibited in the normal sample overlay, beginning with the outer-most spots and progressing inward. This embodiment can use any number of staging biomarkers (i.e., biomarkers associated with severity of disease) by either increasing or decreasing the number of spots in the pattern or the number of clusters of spots so as to multiply either the number of stages or the number of markers used at each stage. For example, such an embodiment might contain antibodies to markers of increasingly progressive metastatic disease. Alternatively they can be antibodies to any of a number of severity related biomarkers from cancer, neuromuscular, neurological degenerative, and cardiovascular disease, metabolic syndrome or diabetes, or any disease where markers delineate disease progression. Alternatively, the intensities can be measured and normalized to the CEP and their intensities graded by a statistical algorithm for automated diagnostic machines.

This embodiment may use different biomarkers indicating the severity of the disease or this embodiment may use different quantities of the standards where the disease severity is documented to be proportional to the quantity of a particular biomarker present.

Staging of Breast Cancer

In another embodiment, the same design is used as in FIG. 7 with the antibody spot construction for nipple aspirate fluid as follows. There is a characteristic normal nipple aspirate fluid pattern found in each normal breast that is characterized by amounts of proteins that are essentially constant from breast to breast and very similar from individual to individual. The standard pattern is set up to reflect these ratios. That is, a CEP, an UP and a Down DEP are chosen that are dramatically different from normal to cancerous breasts, with staging characteristics as well.

Both breasts are tested and the presence of cancer or pre-cancerous conditions and their staging are determined on an individual breast basis. Such a technique will be able to localize the lesions to the breast from which the NAF is taken, stage, and monitor the disease. In addition, by using ductal lavage, one can even localize the problem to individual ducts within the breast. As in FIG. 7, there would be normal and stage patterns for each breast and the patterns can be assessed over time to see if a problem is developing or to evaluate effectiveness of the treatment on an individualized basis.

Although the present invention and its advantages have been described in detail, it should be understood that various changes, substitutions and alterations can be made herein without departing from the spirit and scope of the invention as defined by the appended claims.

What is claimed is:

1. A method for identifying the presence of a protein expression pattern that is characteristic of biological samples taken from an organism having an altered biological state comprising:
a) coating a first and a second solid surface with a plurality of antibodies, wherein said antibodies are antibodies that are reactive to a set of proteins characteristic of a protein expression pattern found in a biological sample taken from an organism having an altered biological state;
b) contacting the first solid surface with a standard sample;
c) washing the standard-contacted first solid surface to remove all protein that is unreacted with the coated antibodies;
d) contacting the second solid surface with a biological sample;
e) washing the sample-contacted second solid surface to remove all protein that is unreacted with the coated antibodies;
f) creating a first digital image of the washed first solid surface and a second digital image of the washed second solid surface;
g) assigning a first color to the first digital image and a second color to the second digital image, wherein an intensity of the first and second colors are proportional to a concentration protein bound to the antibodies;
h) overlaying the first and second digital images; and
i) analyzing the overlaid images to determine if the biological sample was from an organism having the altered biological state.

2. The method of claim 1, wherein the antibodies include an antibody directed against a protein that is up-regulated in the altered biological state.

3. The method of claim 1, wherein the antibodies include an antibody directed against a protein that is down-regulated in the altered biological state.

4. The method of claim 1, wherein the antibodies include an antibody directed against a constitutively expressed protein.

5. The method of claim 4, wherein the antibodies further include an antibody directed against a protein that is up-regulated or down-regulated in the altered biological state.

6. The method of claim 1, wherein the antibodies include an antibody directed against a protein that is known to increase proportionally to the severity of the altered biological state.

7. The method of claim 1, wherein the antibodies include an antibody directed against a protein that is known to decrease proportionally to the severity of the altered biological state.

8. The method of claim 1, wherein the altered biological state is breast cancer.

9. The method of claim 1, wherein the altered biological state is a response to an environmental insult.

10. The method of claim 1, wherein the altered biological state is an increased risk of heart attack.

11. The method of claim 1, wherein the standard sample includes known quantities of a plurality of protein standards.

12. The method of claim 11, wherein the protein standards are associated with the altered biological state.

13. The method of claim 1, wherein the first and second digital images are created using an image analyzer.

14. The method of claim 1, wherein a mixture of the first and second color is visually distinguishable from the first and second colors.

15. The method of claim 1, such that the detection of a single color in the overlaid first and second images indicates that the biological sample was obtained from an organism that does not express the altered biological state.

16. The method of claim 1, such that the detection of a mixture of colors in the overlaid first and second images indicates that the biological sample was obtained from an organism expressing the altered biological state.

17. An assay method for an altered biological state comprising:
a) coating a first and a second solid surface with a plurality of antibodies, wherein each antibody reacts with an antigenic determinant in a protein associated with an altered biological state;
b) contacting the first solid surface with a standard sample;
c) contacting the second solid surface with a biological sample;
d) washing unreacted protein from the standard-contacted first solid surface and the sample-contacted second solid surface;
e) staining the washed first solid surface with a first reporter molecule and the washed second solid surface with a second reporter molecule, wherein the first reporter molecule and the second reporter molecule are visually distinguishable from each other;
f) overlaying the first and second stained solid surfaces; and
g) analyzing the overlaid stained surfaces to determine if the biological sample was from an organism having the altered biological state.

18. The method of claim 17, wherein the antibodies include an antibody directed against a protein that is up-regulated in the altered biological state.

19. The method of claim 17, wherein the antibodies include an antibody directed against a protein that is down-regulated in the altered biological state.

20. The method of claim 17, wherein the antibodies include an antibody directed against a constitutively expressed protein.

21. The method of claim 20, wherein the antibodies further include an antibody directed against a protein that is up-regulated or down-regulated in the altered biological state.

22. The method of claim 17, wherein the antibodies include an antibody directed against a protein that is known to increase proportionally to the severity of the altered biological state.

23. The method of claim 17, wherein the antibodies include an antibody directed against a protein that is known to decrease proportionally to the severity of the altered biological state.

24. The method of claim 17, wherein the standard sample includes known levels of a plurality of protein standards.

25. The method of claim 24, wherein the protein standards are associated with the altered biological state.

26. A kit for screening biological samples to determine the biological samples relationship to an altered biological state comprising:
a) a first solid surface coated with a plurality of individual antibody spots, wherein each antibody reacts with an antigenic determinant of a protein associated with an altered biological state;
b) a second solid surface coated with a plurality of individual antibody spots, wherein a number of the antibody spots on the second solid surface are substantially similar to the antibody spots on the first solid surface;
c) a standard sample;
e) a first reporter molecule;
f) a second reporter molecule; and
g) means for analyzing an overlay of the first coated solid surface reacted with the standard sample and the first reporter molecule with the second coated solid surface reacted with a biological sample and the second reporter molecule.

27. The method of claim 26, wherein the standard sample includes known quantities of a plurality of protein standards.

28. The method of claim 27, wherein the protein standards are associated with the altered biological state.

29. The method of claim 1, wherein the first reporter molecule is a first stain and the second reporter molecule is a second stain, wherein the first stain is visually distinguishable from the second stain.
